(19) United States
(12) Patent Application Publication
(10) Pub. No.: US 2014/0081230 A1
(43) Pub. Date: Mar. 20, 2014

(54) ABSORBENT PRODUCTS WITH IMPROVED DRYNESS

(71) Applicant: John D. LITVAY, Smyrna, GA (US)

(72) Inventor: John D. LITVAY, Smyrna, GA (US)

(21) Appl. No.: 14/031,203

(22) Filed: Sep. 19, 2013

Related U.S. Application Data
(60) Provisional application No. 61/702,996, filed on Sep. 19, 2012.

(51) Int. Cl. A61F 13/53

(52) U.S. Cl. CPC A61F 13/53
USPC 604/372

(57) ABSTRACT

The embodiments provides absorbent garments, such as a disposable diaper, incontinent pad, sanitary napkin, and the like, that has an absorbent core that provides for long term dryness or an improvement in Rewet Values. Such new absorbent garments can be constructed with absorbent cores containing synthetic fibers and super absorbent particulates.

### CROSS-REFERENCE TO RELATED APPLICATION

This application claims benefit to U.S. Provisional application No. 61/702,996 filed Sep. 19, 2013, the contents of which are incorporated by reference herein.

### BACKGROUND

1. Field of the Invention

Embodiments include absorbent products having improved dryness. Specifically, the embodiments pertain to absorbent products that utilize a pulp less core and can thereby eliminate the previously required or desired layers such as the hygrophillic acquisition or transfer layers and/or products that combine the cover sheet and core wrap into a single hybrid cover wrap producing a product with improved dryness at a lower cost.

2. Description of Related Art

Absorbent products, such as baby diapers have a 90 to 95% market penetration in the United States and Europe. The levels of market penetration in some of the emerging market countries such as China and India, however, is less than 5%. Part of the reason for this low level of penetration is the relative high cost of these products in those markets.

Manufacturers of absorbent products in the emerging market countries face an enormous challenge in trying to find ways to produce low cost effective products. The cost of raw materials can comprise up to seventy to eighty percent of the cost to manufacture such products. Production of functional absorbent products usually requires a minimum amount of conventional materials, including absorbent components, and leakage-resistant materials. Reducing the amount of material used beyond these minimums is generally believed to greatly diminish the product's functionality.

Absorbent products such as baby diapers, feminine hygiene pads and adult incontinent products typically are made of several different types of materials. These products usually include a permeable non-woven top sheet, an impermeable back sheet and an absorbent core positioned between the top sheet and the back sheet. The absorbent core typically consists of wood fluff and a water-absorbing polymer, which most commonly is prepared from monomers of acrylic acid. This water-absorbing polymer, referred to as super absorbent polymer (SAP), can constitute anywhere from about 20% up to 50% of the material cost of the product. In addition, availability of SAP fluctuates, and in those times in which there is a shortage of SAP, the costs can be much higher. SAP comes in two distinct classes: polyacryamide based and starch based versions. Therefore it would be desirable to have absorbent products or product designs that could utilize either SAP while maintaining performance and optimizing to the lowest cost position depending upon market condition. Wood-based fluff can account for an additional 10-20% of the material cost of the product.

Conventional absorbent garments having wood-based fluff cores tend to release moisture under pressure over the long term. Baby diapers in particular and some adult incontinent diapers may be worn for long periods of time (2 to 8 hours). Wood-based fluff over that period of time can release moisture through pressure or evaporation. Wood-based fluff does not actually absorb the liquid like SAP. The long term comfort of the wearer therefore is compromised due to the release of this moisture.

Accordingly, there is a need in the art for a cost effective absorbent garment having an improved long term dryness profile that will provide for a much more comfortable and hygienic product.

The description herein of certain advantages and disadvantages of known elements of absorbent garments, methods, and systems is not intended to limit the scope of the present invention to either their inclusion or exclusion. Indeed, certain embodiments may include one or more known elements without suffering from the disadvantages described herein.

### SUMMARY

Various features of the embodiments provide an absorbent garment, such as disposable diapers, adult incontinent pads, sanitary napkins, and the like, that include a pulp less absorbent core that provides improved dryness while eliminating costly layers of construction such as the acquisition or transfer layer and/or that combine the top sheet and core wrap layers into a single hybrid layer.

The above and other features and advantages of the embodiments will become apparent to those having ordinary skill in the art from a review of the following detailed description of the preferred embodiments.

### DETAILED DESCRIPTION OF DRAWINGS

Various features and embodiments described herein will be described with reference to the following non-limiting drawings, in which:

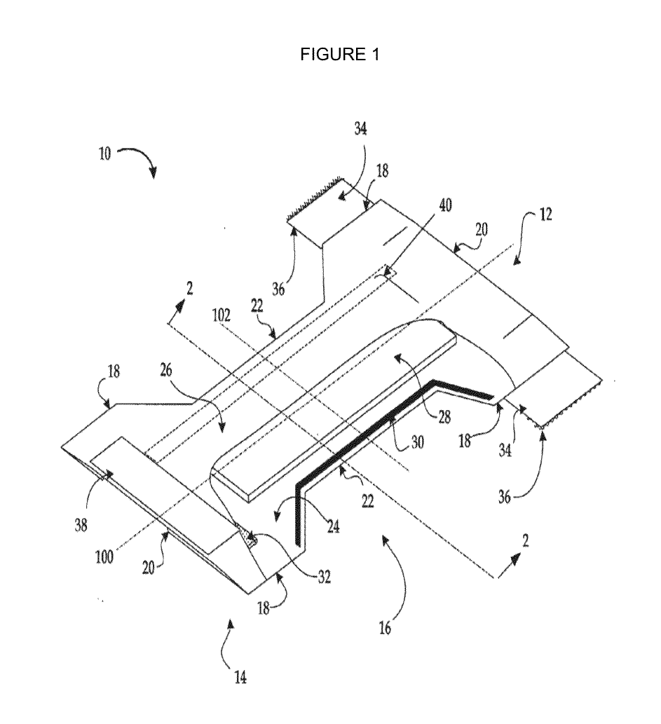

FIG. 1 illustrates a top view of a diaper having an absorbent synthetic core;

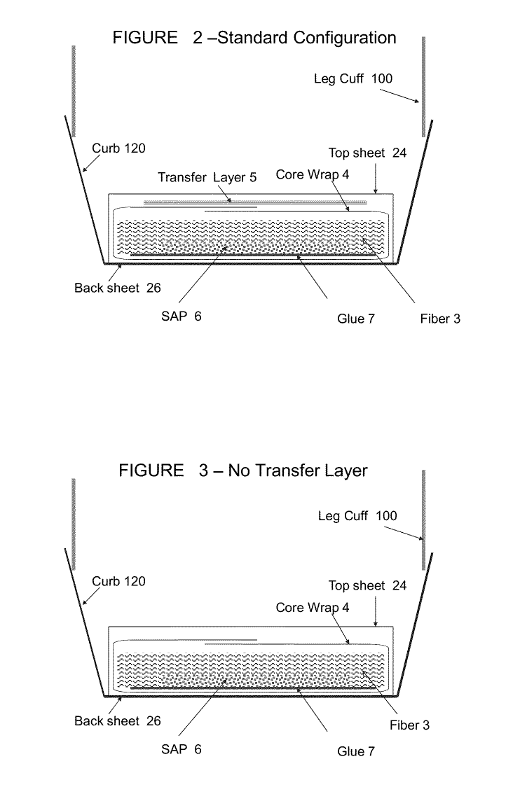

FIG. 2 illustrates a cross-sectional view of an absorbent core showing the synthetic fibers, SAP, glue, standing leg gathers, transfer layer, core wrap, backsheet and topsheet;

FIG. 3 illustrates a cross-sectional view of the absorbent core of FIG. 2 above without the transfer layer or acquisition layer;

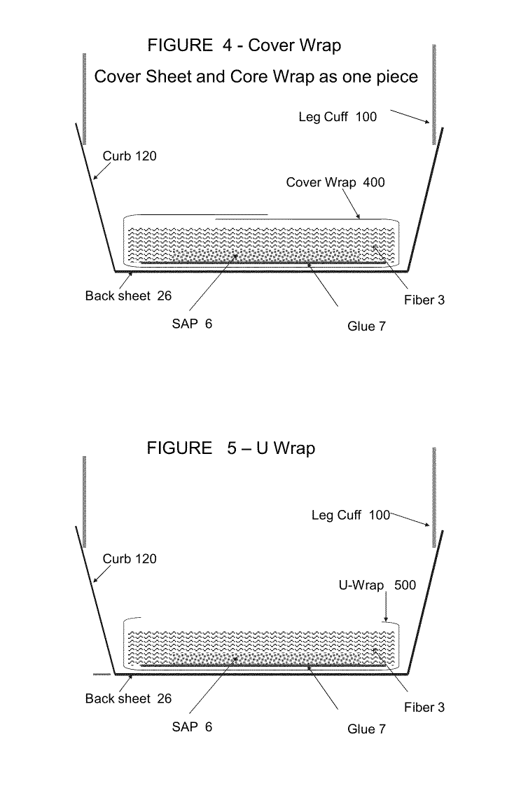

FIG. 4 illustrates a cross-sectional view of the absorbent core of FIG. 2 above but with the transfer layer removed and a single core wrap sheet replacing the topsheet (cover sheet) and the core wrap;

FIG. 5 illustrates a cross-sectional view of the absorbent core of FIG. 2 above but with the transfer layer removed and a single core wrap sheet replacing the topsheet (cover sheet) and the core wrap in the U wrap configuration; and

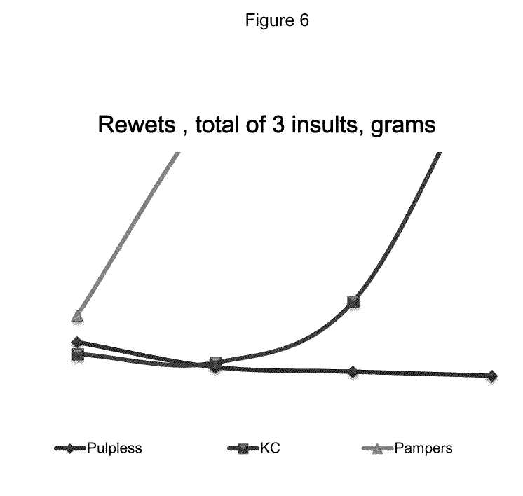

FIG. 6 is a graph showing the results of the comparative tests carried out in the examples.

### DETAILED DESCRIPTION OF INVENTION

Embodiments described herein relate to absorbent garments with improved long term (greater than one hour) dryness or rewet values, when compared to conventional absorbent garments made with wood-based fluff and SAP or to a pulp-less product manufactured using the standard configuration including the use of a transfer layer, topsheet and core wraps. Throughout this description, the expression “absorbent garments” or “absorbent products” or “absorbent articles” denote disposable diapers, incontinent pads, sanitary napkins, adult incontinence garments, absorbent surgical gowns, absorbent surgical sheets or tissue, and the like. In preferred embodiments, the absorbent core of the preferred absorbent articles comprises synthetic fibers and super absorbent polymer.

While not intending on being bound by any theory of operation, the inventor discovered that absorbent products that include synthetic absorbent cores comprised of SAP have a superior long term dryness or rewets when compared to absorbent products having conventional cores that include SAP in a wood-based fluff. The inventor also has discovered that removal of the acquisition or transfer layer has the unexpected benefit of lowering rewet and improving dryness, especially when removed on garments that include a pulpless absorbent core. In a similar manner, the inventor discovered, again unexpectedly, that combining the topsheet and core wrap layers into a single hybrid layer lowers rewet values and improves dryness.

Throughout this description, the expressions “upper layer,” “lower layer,” “above” and “below,” which refer to the various components included in the absorbent composite and absorbent core embodiments (including the layers surrounding the absorbent core units) are used merely to describe the spatial relationship between the respective components. The upper layer or component “above” the other component(s) need not always remain vertically above the core or component(s), and the lower layer or component “below” the other component(s) need not always remain vertically below the core or component(s). Indeed, embodiments include various configurations in which the core may be folded in such a manner that the upper layer ultimately becomes the vertically highest and vertically lowest layer at the same time. Other configurations are contemplated within the context of the present embodiments.

The term “component” can refer, but is not limited to, designated selected regions, such as edges, corners, sides or the like; structural members, such as elastic strips, absorbent pads, stretchable layers or panels, layers of material, a transfer layer, a fluid handling layer, or the like; or a graphic.

Throughout this description, the term “disposed” or “positioned,” and the expressions “disposed on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the expressions “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any continuous fiber. Tow fibers typically are used in the manufacture of staple fibers, and preferably are comprised of synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present embodiments also encompasses modified tow fibers that have been either surface or internally modified (chemically or otherwise) to improve various desired properties of the fibers (e.g., wicking, etc.) as well as tow fibers produced using re-cycled thermoplastic materials.

Throughout this description, the expression “super absorbent polymer” (“SAP”) or “super absorbent material” refers to any polymeric material that is capable of absorbing large quantities of fluid by forming a hydrated gel as well as starch based polymers. Super absorbent polymers are well-known to those skilled in the art as substantially water-insoluble, absorbent polymeric compositions that are capable of absorbing large amounts of fluid (e.g., 0.9% solution of NaCl in water, or blood) in relation to their weight and forming a hydrogel upon such absorption. Super absorbent polymers also can retain significant amounts of water under moderate pressures. Super absorbent polymers generally fall into three classes, namely, starch graft copolymers, cross-linked carboxymethylcelluose derivatives, and modified hydrophilic polyacrylates. Examples of such absorbent polymers are hydrolyzed starch-acrylonitrile graft copolymer a neutralized starch-acrylic acid graft copolymer, a saponified acrylic acid ester-vinyl acetate copolymer, a hydrolyzed acrylonitrile copolymer or acrylamide copolymer, a modified cross-linked polyvinyl alcohol, a neutralized self-cross-linking polyacrylic acid, a cross-linked polyacrylate salt, carboxylated cellulose, and a neutralized cross-linked isobutylene-maleic anhydride copolymer.

Throughout this description, “dryness ” or “rewet” denotes the value determined by first measuring the rewet values of an absorbent garment at various time intervals (30, 60, 120 minutes), in which the rewet value are the aggregated value of 3 insults values of 100 mls, each of 1.0% saline solution collected at the above times of 30, 60, and 120 minutes. Each insult produces a rewet value, the aggregated total of the three insults results in the total rewet value.

Embodiments of the invention also include those in which the absorbent garments includes a “synthetic absorbent core,” and experience lower total rewet values as the hygophillic transfer layers and/or topsheet and core wrap layers are eliminated from the product design. As used throughout this description, the expression “synthetic absorbent core” denotes an absorbent core comprised of SAP and synthetic fibers (preferably tow fibers), and substantially no wood-based fluff pulp. Substantially no wood-based fluff pulp means less than 2% by weight fluff, preferably less than about 0.5% by weight, and most preferably, no fluff pulp.

The embodiments now will be described with reference to the attached drawings illustrating preferred embodiments. Some of the features that appear in more than one figure do not necessarily have the same reference number in each Figure.

FIG. 1 is a partially cut away depiction of an exemplary embodiment of an absorbent garment 10 (preferably a disposable absorbent garment). The embodiment shown in FIG. 1 is an infant's diaper. This depiction, however, is not intended to limit the preferred embodiments, and a person having ordinary skill in the art will appreciate that the preferred embodiments cover other types of absorbent articles. For simplicity, the preferred embodiments will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral (or transverse) axis 102 of the garment is the dimension corresponding to the side-to-side dimension of the user. The longitudinal axis 100 and the transverse axis 102 make up the longitudinal plane of the garment.

In use, the embodiments comprise a garment 10 having a pant-like configuration with a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body, when the garment is worn. The first and second waist regions 12, 14, may correspond to the front and back of the wearer's body, respectively, depending on whether garment 10 is attached in front of or behind the subject wearer. The first and second waist regions may be joined together at or near their lateral edges 18, causing the longitudinally distal edges 20 of the garment 10 to form the perimeter of a waist opening. A crotch region 16 extends between the first and second waist regions, 12, 14, and the crotch edges 22 form the perimeter of a pair of leg openings, when the garment 10 is placed on a subject wearer.

The garment preferably comprises a top sheet 24, and a back sheet 26. When the garment 10 is being worn, the top sheet 24 faces the wearer's body, and the back sheet 26 faces away from the wearer. An absorbent core 28 preferably is positioned between at least a portion of the top sheet 24 and the back sheet 26.

A feature of an embodiment may further comprise various additional features. One or more pairs of elastic gathers 30 (leg elastics) may extend adjacent the crotch edges 22. The garment 10 also may comprise one or more waste containment systems, such as inboard standing leg gathers 40, which preferably extend form the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of waist elastic material 32, such as elastic waist foam or other elastically extensible material, which help contract the garment around the wearer's waist, providing improved fit and leakage prevention. In addition, the ear portions of the garment, e.g., those portions immediately adjacent lateral edges 18 and extending to crotch edges 22, can be comprised entirely or only partially of elastically extensible material (not shown).

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise in whole or in part an elastically extensible material (not shown), and may be designed to stretch around a wearer's waist to provide improved fit, comfort, and leakage protection. Such tabs 34 may be used in conjunction with, or in lieu of, waist elastic material 32, such as foam, or other elastically extensible materials.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14. For example, in one embodiment, the fastening mechanism is a hook and loop fastener in which one fastening element is a hook portion, and a corresponding target device is a loop portion of the hook and loop fastener, or the target device may be the backsheet itself. In another embodiment, the fastening mechanism is a tape fastener system in which one fastening element is an adhesive tape, and a corresponding target device is a tape receiving surface. Other fastening systems may be used in the embodiments, so long as they are capable of fastening the garment 10 about the wearer.

Although not shown in the drawings, the absorbent garment 10 also may include grips attached along the distal edges of each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached”, “joined”, “associated”, and similar terms encompass configurations in which a first part is directly joined to a second part by affixing the first part directly to the second part, by indirectly joining the first part to the second part through intermediate members, by fixing the relative positions of various parts by capturing parts between other parts, or by integrally forming the first and second parts. Persons having ordinary skill in the art will appreciate that various methods or combinations of methods may be used to securely join, attach, or otherwise associate the respective parts of the garment 10 to one another.

The top sheet 24 and back sheet 26 may be constructed from a wide variety of materials known in the art. The embodiments are not intended to be limited to any specific materials for these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10. The back sheet 26 may be covered with a fibrous, non woven fabric such as is disclosed, for example, in U.S. Pat. No. 4,646,362 issued to Heran et al., the disclosure of which is hereby incorporated by reference in its entirety.

The top sheet 24 preferably is moisture-pervious, or fluid-permeable, thereby allowing fluids and other body exudates to flow there-through. The moisture-pervious top sheet 24 can be comprised of any suitable relatively liquid-pervious material known in the art that permits passage of liquids. Non-woven liner sheet materials are exemplary because such materials readily allow the passage of liquids to the underlying absorbent core 28. Examples of suitable liner sheet material include non-woven spun bond or carded webs of polypropylene, polyethylene, nylon, polyester, and blends of these materials.

The top sheet 24 and back sheet 26 can be shaped and sized according to the requirements of each of the various types of absorbent garments, or to accommodate various user-sizes. In an embodiment in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

The underlying structure beneath the top sheet 24 may include, depending on the diaper construction, various combinations of elements, but in each embodiment, it is contemplated that the absorbent garment preferably will include an absorbent core 28 comprising synthetic fibers and SAP. In conventional absorbent articles, an additional layer, known as a transfer layer or acquisition layer, or fluid distribution layer (shown as transfer layer 5 in FIG. 2), typically is provided to improve acquisition of fluids. The inventor has found that removing the fluid distribution, or acquisition, or transfer layer from the absorbent garment provides an unexpected decrease in rewet and an unexpected improvement in dryness.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a limited amount of material. The absorbent core may be associated with the top sheet 24, back sheet 26, or any other suitable part of the garment 10 by any method known in the art, in order to fix the absorbent core 28 in place. Persons of ordinary skill in the art are capable of designing and wrapping a suitable absorbent core 28 of the embodiments, using the guidelines provided herein.

FIG. 2 shows a cut away cross-section of absorbent core 1. FIG. 2 illustrates a synthetic fiber matrix 3, a wrap material 4 and an acquisition layer or transfer layer 5. The absorbent core 1 can be comprised of any synthetic material known in the art and SAP. Preferably, the synthetic fiber matrix 3 includes thermoplastic hygrophobic materials which can be from either virgin or re-cycled sources such as polypropylene, polyethylene terephthlate (PET), etc. “Tow” or “tow fibers” as they are used herein, include synthetic materials such as polyolefins, rayon, polycarbonates, and cellulose acetate. Polyolefins include polypropylene, polyethylene, polybutylene, and mixtures and copolymers thereof. The curb 120 illustrated in FIG. 2 should be at a height so as to create the necessary volumetric capacity or pool volume to retain all the liquid while the SAP 6 absorbs the liquid. It is important that the standing leg gather and hence curb height be above the CRC maximum capacity swollen core volume to prevent contact with the core and aid in controlling leakage. The curb height should not be significantly greater than the swollen core height so that material is wasted and the “fit” of the product is compromised. In preferred embodiments, the curb height can be in the range of 0 to 5 mm, 5 to 10 mm, 10 to 15 mm, 15 to 20 mm, 20 to 25 mm, 25 to 30 mm, or 30 to 35 mm.

FIG. 3 shows the cut away cross-section of the absorbent core of FIG. 2 without the acquisition or transfer layer 5.

FIG. 4 shows the cut away cross-section of the absorbent core of FIG. 2 without the acquisition or transfer layer 5, and the topsheet 24 and the core wrap 4 are replaced with a single hybrid cover wrap 400. The hybrid cover wrap can be comprised of a phillic non-woven, microporous film or topside apertured film. This core design is most suited for sonic bonding, pressure embossing or heated embossing, in that it contains a minimal number of layers while containing the SAP and all the layers can be composed of thermoplastic materials that are amenable to such treatments. The use of re-cycled plastics materials for the tow fibers can have a desirable effect on such processing due to the lower crystallinity and melt points usually encounter with such sources of materials.

FIG. 5 shows the cut away or cross-section of the absorbent core of FIG. 2 without the acquisition or transfer layer, and the cover wrap, resulting in a U-Wrap design 500 which therefore exposes the hygrophobic tow fibers to the skin of the wearer, or in the case of a fluff pulp product, the fiber SAP mixture. The design is not feasible at present to produce but can be used to illustrate and support the concepts described herein.

The absorbent core can be prepared by methods known in the art. For example, the feeding system disclosed in U.S. Pat. No. 6,923,926, which is incorporated herein by reference in its entirety, can be used to prepare the synthetic fiber matrix that includes the tow material and SAP. It also is understood that the manufacture of the absorbent core can be done off-line and separate from the manufacturing of the remainder of the absorbent garment components. It is possible, for example, to have one absorbent core-forming unit supply several diaper manufacturing machines.

Combinations of certain types of SAP applied in certain patterns within a synthetic core may result in an absorbent core that provides absorbent garments with excellent long-term dryness. To establish which combinations are most effective, the present inventor developed a protocol to determine the dryness or rewets so that absorbent articles prepared in accordance with the embodiments could be compared to conventional products with respect to long-term dryness and rewet. The dryness can be determined by measuring the rewet values of an absorbent garment in various time intervals (30, 60, 90, or 120 minutes) in which the time intervals were measured for 3 insults of 100 mls each of 1.0% saline solution. Each of the three resultant rewet values were recorded and the aggregate of the three insults are reported.

The embodiments now will be described with reference to the following non-limiting example.

### EXAMPLES

The following 1^{st} insult procedure was used to measure the rewet values over each time interval:
A diaper was laid open with the absorbent core and transfer layer (if present) facing up.
100 mls of a 1.0% saline solution was applied to the center of the absorbent core of the diaper.
After a time interval of 30 minutes, a known “dry” weight (g) of blotter papers (90 mm×140 mm) were applied to the insult area under a 4800 g weight (90 mm×140 mm) for 120 seconds.
The blotter papers were then removed and weighed resulting in the wet weight (grams).
The rewet value was then calculated by subtracting the wet weight (g) from the “dry” weight (g). Wet weight (grams)−Dry weight (grams)=1^{st} Insult Rewet.
This was then repeated two more times at 60 and 120 minutes (2^{nd} and 3^{rd} Rewets) and the aggregate total of the three Insult Rewets was reported as the Rewet Value.

Various products were prepared in accordance with the embodiments shown in FIGS. 2-5, and were compared to the following 2 commercially available diapers containing wood-based fluff pulp/SAP absorbent cores. The first commercial product was a HUGGIES branded product (from Kimberly Clark, Saudi Arabia) having about 10 grams of SAP and 15 grams of fluff pulp, in which the acquisition layer had a basis weight of approximately 50 grams and did not run the full length of the core. The second commercial product (Fluff #2) was a PAMPERS product (from P&G Saudia Arabia) having about 10 grams of SAP and 15 grams of fluff pulp, and the non-woven acquisition layer (basis weight of about 40 grams) also did not run the full length of the core.

The first absorbent garment, Pulpless A (having a standard design as shown in FIG. 2) comprised an acquisition layer comprised of a non-woven transfer layer of approximately 35 grams purchased from Pantex. The acquisition layer did not run the complete length of the absorbent core. The absorbent core included about 13.5 grams of SAP (HySorb purchased from BASF, Germany) having a Centrifuge Retention Capacity (CRC) of about 29.

The second absorbent garment Pulpless B (having no transfer layer as shown in FIG. 3) comprised the same product as Pulpless A above but with the transfer layer removed using standard laboratory practices know to those skilled in the art of: slicing open on three sides the topsheet and peeling back the topsheet to reveal the transfer layer. The transfer layer was then removed using forceps with slight heat or solvent if required. Once the transfer layer was removed, the topsheet was replaced and tacked down.

The third absorbent garment Pulpless C (having a cover wrap as shown in FIG. 4) was the same product as Pulpless A mentioned above but with the transfer layer and core wrap removed using the same techniques described above. The topsheet was replaced to simulate a cover wrap (although not optimized, it was able to simulate or demonstrate the embodiments).

The fourth absorbent garment, Pulpless D (having a U-wrap as shown in FIG. 5) was a similar sample as Pulpless A above except that all layers of materials above the core fiber were removed, so that testing could be conducted on the “tow or fiber” itself.

The fifth through eighth absorbent garments, KC-A thru KC-D comprised HUGGIES samples similar to those described above that were treated in a like manner to the Pulpless samples so as to yield garments similar to those represented in FIGS. 2-5 and of a like design as the Pulpless garments A-D

The ninth thru twelfth absorbent garments, PAMPERS-A thru PAMPERS-D comprised PAMPERS samples similar to those described above that were treated in a like manner to those Pulpless samples so as to yield garments similar to those represented in FIGS. 2-5 and of a like design as the Pulpless garments A-D.

TABLE 1
The Rewet Values from Three Insults

3 insults (grams)

| | | B-No | C-Cover | D-U |
|---|---|---|---|---|
| | A-Standard | TL | Wrap | Wrap |
| Pulpless (A-D) | 6.5 | 2.1 | 1.3 | 0.6 |
| KC (A-D) | 4.4 | 2.9 | 13.7 | 58.1 |
| Pampers (A-D) | 11.2 | 49.1 | 71.5 | 92.0 |

From Table 1 the following observations can be made
1) For the Pulpless garments as the phillic transfer layer, topsheet or core wrap are removed or eliminated from the design the rewets drop or dryness improves. This is in contradiction for the wood pulp/SAP containing garments where it was found that in general the removal of these layers causes an increase in rewet or a loss in dryness.
2) Lastly the garment with the overall lowest Rewets are the two Pulpless products C (1.3 grams) and D (0.6 grams).

FIG. 6 is a graph illustrating the above conclusions.

What is claimed is:

1. An absorbent product comprising:
a) a liquid permeable top sheet;
b) an absorbent core comprising synthetic fibers and super absorbent particulate; and
c) a back sheet;
wherein the rewet values of the absorbent product are lower than the rewet values for the same absorbent product that contains an acquisition layer or transfer layer.

2. The absorbent product of claim 1, wherein the synthetic fibers are selected from the group consisting of rayon, polypropylene, cellulose acetate, or polyethylene.

3. The absorbent product of claim 1, wherein the synthetic fibers are produced using virgin resins.

4. The absorbent product of claim 1, wherein the synthetic fibers are produced using recycled materials.

5. The absorbent product of claim 1, wherein the super absorbent particulate are made from acrylic acid or starch based polymers.

6. The absorbent product of claim 1, further comprising a cover wrap around the absorbent core.

7. The absorbent product of claim 6, wherein the cover wrap comprises a phillic non-woven, microporous film, or topside apertured film.

8. The absorbent product of claim 1, wherein the absorbent core is sonic bonded, pressure or heat embossed.

9. The absorbent product of claim 1, further comprising a curb.

10. The absorbent product of claim 1, wherein the curb has a curb height of greater than 15 mm.

11. An absorbent product comprising:
a) a liquid permeable top sheet;
b) an absorbent core comprising synthetic fibers and super absorbent particulate; and
c) a back sheet;
wherein the rewet values of the absorbent product are lower than the rewet values for the same absorbent product that contains an acquisition layer or transfer layer and a separate top sheet and wrapping material surrounding the absorbent core.

12. The absorbent product of claim 11, wherein the synthetic fibers are selected from the group consisting of rayon, polypropylene, cellulose acetate, or polyethylene.

13. The absorbent product of claim 11, wherein the synthetic fibers are produced using virgin resins.

14. The absorbent product of claim 11, wherein the synthetic fibers are produced using recycled materials.

15. The absorbent product of claim 11, wherein the super absorbent particulate are made from acrylic acid or starch based polymers.

16. The absorbent product of claim 11, further comprising a cover wrap around the absorbent core.

17. The absorbent product of claim 16, wherein the cover wrap comprises a phillic non-woven, microporous film, or topside apertured film.

18. The absorbent product of claim 11, wherein the absorbent core is sonic bonded, pressure or heat embossed.

19. The absorbent product of claim 11, further comprising a curb.

20. The absorbent product of claim 19, wherein the curb has a curb height of greater than 15 mm.
